SEQUENCE LISTING (9 sequences)

SEQ ID NO: 1 - DNA, 29 bp, Artificial Sequence
  Description of Artificial Sequence Synthetic oligonucleotide
aaaaaagacgagattcgatcggagtaaaa

SEQ ID NO: 2 - DNA, 29 bp, Artificial Sequence
  Description of Artificial Sequence Synthetic oligonucleotide
ttttactccgatcgaatctcgtctttttt

SEQ ID NO: 3 - DNA, 29 bp, Artificial Sequence
  Description of Artificial Sequence Synthetic oligonucleotide
aaaaaagacgagattcgatcggagtaaaa

SEQ ID NO: 4 - DNA, 33 bp, Mus sp.
aaaaagcccttctattgaaacacagatacaaaa

SEQ ID NO: 5 - DNA, 33 bp, Mus sp.
ttttgtatctgtgtttcaatagaagggcttttt

SEQ ID NO: 6 - DNA, 33 bp, Mus sp.; identical to SEQ ID NO: 4

SEQ ID NO: 7 - DNA, 33 bp, Homo sapiens
aaaaagcccttctattaaaacacagacacaaaa

SEQ ID NO: 8 - DNA, 33 bp, Homo sapiens
ttttgtgtctgtgttttaatagaagggcttttt

SEQ ID NO: 9 - DNA, 33 bp, Homo sapiens; identical to SEQ ID NO: 7

(19) United States
(12) Patent Application Publication
(10) Pub. No.: US 2019/0136302 A1
(43) Pub. Date: May 9, 2019

(54) METHODS FOR DETECTING OLIGONUCLEOTIDES IN A SAMPLE

(71) Applicant: THE REGENTS OF THE UNIVERSITY OF CALIFORNIA, Oakland, CA (US)

(72) Inventors: Ameae M. WALKER, Riverside, CA (US); Mary Y. LORENSON, Riverside, CA (US)

(73) Assignee: THE REGENTS OF THE UNIVERSITY OF CALIFORNIA, Oakland, CA (US)

(21) Appl. No.: 16/306,807

(22) PCT Filed: Jun. 6, 2017
(86) PCT No.: PCT/US17/36153

§ 371 Date: Dec. 3, 2018

Related U.S. Application Data
(60) Provisional application No. 62/346,292, filed on Jun. 6, 2016.

(51) Int. Cl. C12Q 1/6834; C12Q 1/6851; C12Q 1/6886

(52) U.S. Cl. CPC C12Q 1/6834; C12Q 1/6851; C12Q 1/6886; C12Y 301/03001; C12Q 2600/166; C12Y 302/01023; C12Y 111/01007; C12Q 2600/112

(57) ABSTRACT

Certain embodiments of the invention provide a method (i.e., Enzyme Linked Oligonucleotide Sorbent Assay (ELOSA)) for the detection and/or quantification of a test oligonucleotide (e.g., a small oligonucleotide) in a test sample, such as a biological fluid, comprising: a) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture; b) contacting the reaction mixture with a substrate that specifically binds to the enzyme, thereby generating an enzyme-substrate reaction product; and c) measuring the concentration of the enzyme-substrate reaction product, so as to detect and/or quantify the test oligonucleotide.

### RELATED APPLICATION

This application claims the benefit of priority of U.S. Provisional Application Ser. No. 62/346,292 filed on Jun. 6, 2016, which application is incorporated by reference herein.

### GOVERNMENT FUNDING

This invention was made with Government support under Grant No. BC132946 awarded by the Department of Defense. The Government has certain rights in this invention.

### BACKGROUND OF THE INVENTION

Oligonucleotide therapeutics, including splice-modulating oligonucelotides (SMOs), siRNAs, and shRNAs, are increasingly being used to treat a variety of disease states. Additionally, miRNAs are now being used as diagnostic indicators for cancer, as well as to monitor therapeutic responses. However, current methods for measuring small oligonucleotides, such as capillary electrophoresis (Khan et al., 1997 J Chromatogr B Biomed Sci Appl 702: 69-76) and electrospray mass spectrometry (Griffey et al., 1997 J Mass Spectrom 32:305-313), involve extraction and complicated detection methods not suitable for the clinical setting. A single-stranded DNA-binding fluorophore, such as OliGreen, has also been suggested (Gray G D, Wickstrom E 1997 Antisense and Nucleic Acid Drug Development 7:133-140); however, interference from non-specific fluorescence in serum, especially in tumor-bearing animals, reduced the sensitivity and reliability of the method. While quantitative PCR (qPCR) could also potentially be used to measure such oligonucleotides (Mar-Aguilar et al., 2013 Disease Markers 34:163-169), this method requires relatively expensive equipment, is time consuming, has inherent quality control and reproducibility issues and requires isolation of the oligonucleotide from the biological fluid.

Accordingly, there is a need for new methods for the detection of small oligonucleotides in biological fluids. Specifically, there is a need for inexpensive, efficient, sensitive and reproducible methods for the detection of small oligonucleotides.

### SUMMARY OF THE INVENTION

Described herein are methods for the rapid and sensitive detection of oligonucleotides (e.g., small oligonucleotides). Specifically, the ELOSA (Enzyme-Linked Oligonucleotide-Sorbent Assay) described herein may be used to quantify oligonucleotides in sample, such as serum or other biological fluids (e.g., for monitoring drug delivery, pharmacokinetics, diagnostics and monitoring therapeutic responses) (see, FIG. 1). The assay is simple, sensitive, and does not require laborious extractions or expensive equipment.

Accordingly, certain embodiments of the invention provide a method for the detection and/or quantification of a test oligonucleotide in a test sample comprising:

a) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture;

b) contacting the reaction mixture with a substrate that specifically binds to the enzyme, thereby generating an enzyme-substrate reaction product; and

c) measuring the concentration of the enzyme-substrate reaction product, so as to detect and/or quantify the test oligonucleotide.

Certain embodiments of the invention provide a method for the detection and/or quantification of a test oligonucleotide in a test sample comprising:

a) contacting the test sample with i) a capture reagent bound to a first solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a test reaction mixture;

b) contacting the test reaction mixture with a substrate that specifically binds to the enzyme, thereby generating a test enzyme-substrate reaction product;

c) measuring the concentration of the test enzyme-substrate reaction product;

d) contacting a control sample comprising a predetermined amount of the test oligonucleotide with i) a capture reagent bound to a second solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a control reaction mixture;

e) contacting the control reaction mixture with a substrate that specifically binds to the enzyme, thereby generating a control enzyme-substrate reaction product; and

f) measuring the concentration of the control enzyme-substrate reaction product, wherein the relative concentration of the test enzyme-substrate reaction product to the control enzyme-substrate reaction product indicates the presence and/or quantity of the test oligonucleotide.

Certain embodiments of the invention also provide a kit for detecting and/or quantifying a test oligonucleotide in a test sample comprising:

a) a capture reagent, wherein the capture reagent comprises an oligonucleotide comprising a nucleic acid sequence complementary to the test oligonucleotide;

b) a competition oligonucleotide operably linked to an enzyme, wherein the competition oligonucleotide comprises a nucleic acid sequence complementary to the capture oligonucleotide;

c) a standard test oligonucleotide(s) for calibration

d) a substrate, wherein the substrate is capable of specifically binding to the enzyme to generate an enzyme-substrate reaction product, and wherein the enzyme-substrate reaction product is detectable spectrophotometrically or fluorometrically; and

e) instructions for use.

### BRIEF DESCRIPTION OF THE FIGURES

FIG. 1. Illustration of the ELOSA. An aminated and extended capture oligonucleotide is bound to the ELOSA plate via the amine group (solid line). The capture oligonucleotide is the antisense of the oligo to be tested. The test oligomer (e.g., a splice-modulating oligomer (SMO)) in the sample (dashed line without star in panel 2)) is in competition for binding to the capture oligonucleotide with a peroxidase-labeled oligonucleotide (i.e., the competition oligonucleotide; same base sequence as test oligo but conjugated to horseradish peroxidase, and shown as a dashed line with star), which is present at constant concentration (panel 2). After incubation, a proportion of the peroxidase-labeled oligonucleotide (dependent on the degree of competition) will be bound (panel 3). After washing, peroxidase substrate is then added to generate a colored product (panel 4). To ensure adequate competition between the labeled and unlabeled oligos, the amount of the capture oligomer should be limiting.

FIG. 2. Assay Specificity. In the experiments described in the Examples, the PRLR SMO vivo-morpholino is referred to as Antimaia to distinguish it from the oligonucleotides being used for competition or capture, which are called HRP-PRLR SMO and capture PRLR SMO, respectively. Absorbance levels were recorded using the PRLR SMO Capture Oligonucleotide at increasing concentrations and the HRP-PRLR SMO (diamonds), HRP-PRLR SMO+control (vivo-morpholino control SMO) (squares) or HRP-PRLR SMO+Antimaia (circles). The control SMO did not hybridize to the PRLR SMO Capture Oligo, but Antimaia displaced HRP-PRLR SMO, indicating the specificity of the assay.

FIG. 3. General Applicability of the Assay. Absorbance levels were recorded using the Control SMO Capture Oligonucleotide at increasing concentrations and HRP-control SMO (diamonds), HRP-control SMO+control (vivo-morpholino control SMO) (squares) or HRP-control SMO+Antimaia (i.e., a vivo-morpholino of PRLR SMO) (triangles). The control SMO (5 pmoles) competed with HRP-control SMO (1 pmole) for binding to the Control SMO Capture Oligonucleotide (5 pmoles), whereas Antimaia (i.e., a vivo-morpholino of PRLR SMO) (5 pmoles) had essentially no effect. These results indicate that the assay can be used as a general method to detect oligonucleotides, modified oligomers (e.g., morpholino oligos), and oligos modified for cell penetration (e.g., vivo-morpholino oligos).

FIG. 4. Dose Dependency and Detection Levels. Absorbance levels were recorded using the PRLR SMO Capture Oligonucleotide (2 pmoles), the HRP-PRLR-SMO (0.5 pmole) and varying concentrations of Antimaia (i.e., a vivo-morpholino of PRLR SMO). Increased absorbance was observed in the presence of serum and the assay under these conditions had a sensitivity of 0.25 pmoles with an assayable range from 0.25 to 2 pmoles.

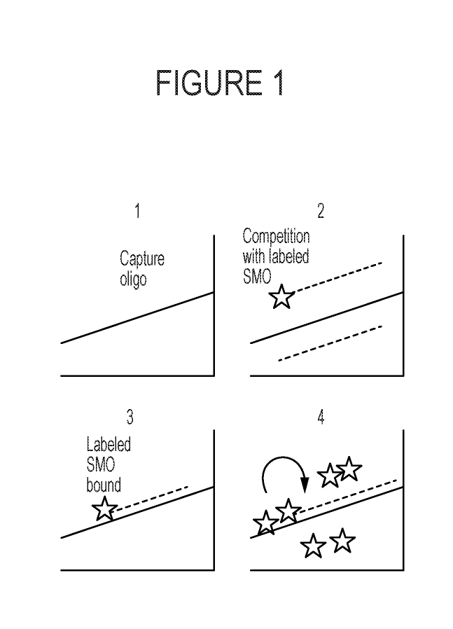

### DETAILED DESCRIPTION

As described herein, a rapid and sensitive assay termed “ELOSA” (Enzyme-Linked Oligonucleotide-Sorbent Assay) has been developed for the detection and quantification of oligonucleotides or modified oligos in a sample, such as a biological fluid (e.g., an oligonucleotide present in picomole or sub-picomole quantities). Prior to this invention, methods for quantification in biological fluids were very cumbersome, time-consuming, involved substantial losses and therefore inaccuracies, and were not suitable for application to the clinic. Oligonucleotide biologicals, including modified oligonucleotides (e.g., vivo-morpholino oligos), splice-modulating DNA oligonucleotides (SMO) and variously-delivered siRNAs are promising as new therapeutics for a number of diseases, including muscular dystrophy, immune disorders, and some cancers. Thus, the ELOSA method could be used to monitor drug concentration and pharmacokinetics. Also, since RNA and DNA fragments could be measured, the method may be applicable to measure infection (e.g., a bacterial infection). Furthermore, measurement of miRNAs in the serum is proving to be useful diagnostically in a variety of cancers. The ELOSA method may therefore be used to screen for cancers and to monitor the effectiveness of a given cancer therapy. In addition, these small oligonucleotides are very widely used in basic research and their quantification is important for the accurate interpretation of experimental results. Accordingly, methods of the invention are applicable to both clinical and basic research settings.

ELOSA utilizes a capture reagent comprising an oligonucleotide (e.g., an aminated capture oligonucleotide for binding to a solid support, such as a 96 well plate), which is antisense to the test oligonucleotide (e.g., a modified oligo) being measured and may be extended with a spacer (e.g., a 12 carbon aliphatic spacer). The spacer allows both binding to the solid support, such as a 96-well plate, and enables hybridization to the test oligo without interference from the solid support. In certain embodiments, the solid support may be a DNA-binding plate, such as those sold by Corning. In the ELOSA, a competition oligonucleotide operably linked to an enzyme (e.g., horseradish peroxidase (HRP)) is used in a known, constant concentration. The HRP-competing oligonucleotide has the same base sequence as the test oligo. The sample containing the test oligo is incubated with the capture reagent, which is bound to the solid support, in the presence of competition oligonucleotide.

After hybridization and washing, competition between the test oligonucleotide (e.g., a modified oligo) and the competition oligonucleotide is measured (e.g., HRP activity may be measured spectrophotometrically or fluorometrically). For example, if HRP is used as the enzyme, there will be a direct correlation between test oligonucleotide and the HRP activity detected: the more of the test oligo that is present, the less of the competition oligonucleotide binds to the capture reagent.

ELOSA does not require extraction of samples from serum or technically sophisticated, expensive equipment, such as in Next Generation Sequencing, and uses equipment common to a research or clinical laboratory (microplate spectrophotometer). One example of the method described herein utilizes the colorimetric peroxidase substrate 3,3′,5,5′-tetramethylbenzidine dihydrochloride (TMB, sold by Sigma chemical company) and hydrogen peroxide to detect two different modified oligos in mouse serum, one of which is a splice modulating oligomer for the prolactin receptor that is a very promising drug for the treatment of breast and ovarian cancer. The ELOSA was concentration dependent (i.e, for the test oligo), specific, and sensitive to 0.25 pmoles, well within the needed range of sensitivity. Thus, ELOSA has broad applicability both in biological research and clinical diagnostics.

### Methods of the Invention

Certain embodiments of the invention provide a method for the detection and/or quantification of a test oligonucleotide (e.g., a modified oligo) in a test sample (e.g., a biological sample, such as a biological fluid, obtained from a test subject, such as a mammal), comprising measuring the concentration of a test enzyme-substrate reaction product in the test sample, wherein the test sample has been contacted by a capture reagent as described herein, a competition oligonucleotide operably linked to an enzyme and a substrate that specifically binds to the enzyme, so as to detect and/or quantify the test oligonucleotide. In certain embodiments, the concentration of the test enzyme-substrate reaction product in the test sample is at least about 1-100% less than the concentration of a control enzyme-substrate reaction product in a control sample (i.e., a negative control sample).

Certain embodiments of the invention provide a method for the detection and/or quantification of a test oligonucleotide (e.g., a modified oligo) in a test sample (e.g., a biological sample, such as a biological fluid, obtained from a test subject, such as a mammal) comprising:

a) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture;

b) contacting the reaction mixture with a substrate that specifically binds to the enzyme, thereby generating an enzyme-substrate reaction product; and

c) measuring the concentration of the enzyme-substrate reaction product, so as to detect and/or quantify the test oligonucleotide.

In certain embodiments of the invention, it is desirable to assay the test sample in parallel with a control sample, which comprises a predetermined amount of the test oligonucleotide. Thus, certain embodiments of the invention provide a method for the detection and/or quantification of a test oligonucleotide (e.g., a modified oligo) in a test sample (e.g., a biological sample, such as a biological fluid) comprising:

a) contacting the test sample with i) a capture reagent bound to a first solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a test reaction mixture;

b) contacting the test reaction mixture with a substrate that specifically binds to the enzyme, thereby generating a test enzyme-substrate reaction product;

c) measuring the concentration of the test enzyme-substrate reaction product;

d) contacting a control sample comprising a predetermined amount of the test oligonucleotide with i) a capture reagent bound to a second solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a control reaction mixture;

e) contacting the control reaction mixture with a substrate that specifically binds to the enzyme, thereby generating a control enzyme-substrate reaction product; and

f) measuring the concentration of the control enzyme-substrate reaction product, wherein the relative concentration of the test enzyme-substrate reaction product to the control enzyme-substrate reaction product indicates the presence and/or quantity of the test oligonucleotide.

In certain embodiments, the predetermined amount of the test oligonucleotide (e.g., a modified oligo) is no test oligonucleotide (i.e., a negative control sample). In such an embodiment, a concentration of the test enzyme-substrate reaction product less than the concentration of a negative control enzyme-substrate reaction product indicates that the test sample comprises the test oligonucleotide.

In certain embodiments, the test sample is further contacted with a buffer, such as a buffer solution, having a pH of about pH 6.5 to about pH 8.5, or about pH 7.0 to about pH 8.0, or about pH 7.5.

As described herein, a “buffer solution” refers to an aqueous solution consisting of a mixture of a weak acid and its conjugate base, or vice versa, and its pH changes very little when a small amount of strong acid or base is added to it. Buffer solutions and buffering agents are known in the art. For example, in certain embodiments, the buffer comprises TrisHCl (e.g., about 0.05 M). In certain embodiments the buffer comprises EDTA (e.g., 1 mM).

In certain embodiments, the methods further comprise obtaining a test sample (e.g., a biological sample) from a test subject (e.g., a mammal, e.g., a human).

In certain embodiments, the methods further comprise binding the capture reagent to a solid support prior to contact with the test sample. To facilitate such binding, the capture reagent is contacted with the solid support under conditions suitable for binding between the two elements to occur. In certain embodiments, the capture reagent is incubated with the solid support under a set of conditions described herein. For example, in certain embodiments the capture reagent is bound to a 96-well Corning DNA plate using 0.05M Na phosphate buffer-pH 8.5, 1 mM EDTA at 4° C.

In certain embodiments, the methods further comprise one or more washing steps. For example, in certain embodiments, the capture reagent bound to the solid support is washed one or more times (e.g., two or three or more times) with a buffer solution (e.g., 0.05 M TrisHCl (pH8); 1 mM EDTA) (i.e., prior to contact with the test sample). In certain embodiments, the capture reagent bound to the solid support is washed with a buffer solution under conditions suitable to remove any unbound capture reagent, prior to being contacted with the test sample. In certain embodiments, the capture reagent bound to the solid support is washed three or more times.

In certain embodiments, the methods further comprise one or more “blocking steps”, wherein the capture reagent bound to the solid support is contacted with a blocking solution that reduces non-specific binding of an oligonucleotide (e.g., for 4 hrs at 37° C.) or other compounds that could compete or interfere with the measurement of the test oligonucleotide (i.e., prior to contact with the test sample). In certain embodiments, the blocking solution comprises bovine serum albumin and at least one buffer solution. In certain embodiments, the blocking solution comprises 3% bovine serum albumin; 0.05M TrisHCl (pH 8.0); and 1 mM EDTA.

In certain embodiments, after the blocking step(s), the methods further comprise one or more washing steps (e.g., two or three or more washing steps). In certain embodiments, after the blocking step(s), the methods further comprise three or more washing steps. For example, in certain embodiments, the capture reagent bound to the solid support is washed after the blocking step(s) one or more times with a buffer solution (e.g., 0.05 M TrisHCl (pH8); 1 mM EDTA). Such washing step(s) are performed under conditions suitable to remove residual blocking solution.

In certain embodiments, the methods further comprise incubating the reaction mixture under conditions and for a time sufficient for binding between the capture reagent and the test oligonucleotide (e.g., a modified oligo) and/or the competition oligonucleotide to occur (i.e., prior to contacting the reaction mixture with the substrate). In certain embodiments, the reaction mixture is incubated under a set of conditions described herein.

In certain embodiments, the methods further comprise one or more washing steps prior to contacting the reaction mixture with a substrate (e.g., two or three or more washing steps). In certain embodiments, the methods further comprise three or more washing steps prior to contacting the reaction mixture with a substrate. For example, in certain embodiments, the reaction mixture is washed one or more times (e.g., three or more times) with a buffer solution (e.g., 0.01 M TrisHCl (pH 7.5); 0.1M NaCl, 0.1% Tween 20 (TBST)). Such washing step(s) are performed under conditions suitable to remove test oligonucleotide(s) or competition oligonucleotide(s) that did not bind to the capture reagent.

As described herein, the methods of the invention comprise contacting the reaction mixture with a substrate that specifically binds to the enzyme. As described herein, the phrase “specifically binds to the enzyme” refers to a substrate that is capable of binding to an enzyme, and once bound, undergoing a chemical reaction to produce an enzyme-substrate reaction product. The reaction mixture and the substrate should be contacted under conditions suitable for catalyzing a chemical reaction between the enzyme and substrate (i.e., to generate the enzyme-substrate reaction product). In certain embodiments, the methods further comprise contacting the reaction mixture and substrate with a secondary agent to generate the enzyme-substrate reaction product. For example, if the enzyme is HRP, hydrogen peroxide may need to be added to catalyze the reaction between HRP and the HRP substrate. The amount of substrate to be added and reaction conditions will be specific to the substrate-enzyme combination selected and can be determined/optimized by one skilled in the art.

As described herein, oligonucleotides, such as modified oligos, are increasingly being used as therapeutics. Accordingly, the concentrations and/or pharmacokinetics of such oligos can be monitored using the methods of the invention. Additionally, the presence/levels of certain miRNAs are now being associated with certain pathological states, such as cancer. Further, the method could be used to detect infection (e.g., bacterial RNA). Thus, certain diseases, disorders or conditions could be diagnosed and/or the effectiveness of a therapy could be monitored using methods of the invention.

Accordingly, certain embodiments of the invention provide a method for diagnosing a disease, disorder or condition (e.g., cancer or a bacterial infection) in a mammal (e.g., a human) comprising:
a) detecting the presence and/or concentration of a test oligonucleotide in a test sample obtained from the mammal by:
   1) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture;
   2) contacting the reaction mixture with a substrate that specifically binds to the enzyme, thereby generating an enzyme-substrate reaction product; and
   3) measuring the concentration of the enzyme-substrate reaction product, so as to detect and/or quantify the test oligonucleotide;
b) diagnosing the mammal with the disease, disorder or condition when the presence or certain concentration of the test oligonucleotide is detected.

In certain embodiments, the method further comprises obtaining a test sample from the mammal.

In certain embodiments, the method further comprises administering a therapeutic agent to the diagnosed mammal. As used herein, the term “therapeutic agent” includes agents that provide a therapeutically desirable effect when administered to an animal (e.g., a mammal, such as a human). The agent may be of natural or synthetic origin. For example, it may be a nucleic acid (e.g., a SMO or siRNA), a polypeptide, a protein, a peptide, or an organic compound, such as a small molecule. The term “small molecule” includes organic molecules having a molecular weight of less than about, e.g., 1000 amu. In one embodiment a small molecule can have a molecular weight of less than about 800 amu. In another embodiment a small molecule can have a molecular weight of less than about 500 amu. In certain embodiments, the therapeutic agent is an anti-cancer agent. In certain embodiments, the anti-cancer agent is a SMO, such as a PRLR SMO, e.g., a PRLR SMO as described herein. In certain embodiments, the therapeutic agent is an antibiotic.

Certain embodiments of the invention also provide a method for evaluating the effectiveness of therapeutic agent in a mammal comprising:
a) detecting the presence and/or concentration of a test oligonucleotide in a first test sample from the mammal by:
   1) contacting the first test sample with i) a capture reagent bound to a first solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a first reaction mixture;
   2) contacting the first reaction mixture with a substrate that specifically binds to the enzyme, thereby generating a first enzyme-substrate reaction product; and
   3) measuring the concentration of the first enzyme-substrate reaction product, so as to detect and/or quantify the test oligonucleotide in the first sample;
b) administering a therapeutic agent to the mammal;
c) detecting the presence and/or concentration of the test oligonucleotide in a subsequent second test sample from the mammal by:
   1) contacting the second test sample with i) a capture reagent bound to a second solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a second reaction mixture;
   2) contacting the second reaction mixture with a substrate that specifically binds to the enzyme, thereby generating a second enzyme-substrate reaction product; and
   3) measuring the concentration of the second enzyme-substrate reaction product, so as to detect and/or quantify the test oligonucleotide in the second sample;
d) determining the effectiveness of the therapeutic agent (e.g., the test oligonucleotide is associated with a disease, disorder or condition and the therapeutic agent would be determined to be effective if the concentration of the test oligonucleotide in the second test sample is less than the concentration of the test oligonucleotide in the first test sample).

In certain embodiments, the disease, disorder or condition is cancer (e.g., breast cancer). In certain embodiments, the disease, disorder or condition is a bacterial infection. In certain embodiments, the disease, disorder or condition is muscular dystrophy. In certain embodiments, the disease, disorder or condition is an immune disorder.

In certain embodiments, the test oligonucleotide is a miRNA. In certain embodiments, the test oligonucleotide is a bacterial nucleic acid (e.g., bacterial RNA or DNA).

Test Oligonucleotide(s) and Sample(s)

The methods described herein may be used to detect and/or quantify an oligonucleotide(s) (e.g., a modified oligo) in a test sample, such as a biological fluid (e.g., present in molar, millimolar, micromolar, nanomolar, picomolar or sub-picomolar concentrations). Such an oligonucleotide is referred to herein as a “test oligonucleotide”. For example, as described in the Examples, the methods of the invention have been used to effectively detect modified oligos present in serum in picomolar/sub-picomolar concentrations.

Thus, in certain embodiments, the concentration of the test oligonucleotide in the test sample is less than about, e.g., 10 mole, 1 mole, 100 millimole, 10 millimole, 1 millimole, 100 micromole, 10 micromole, 1 micromole, 100 nanomole, 10 nanomole, 1 nanomole, 100 picomole, 10 picomole, 1 picomole or 0.1 picomole.

As used herein, a “test sample” may be any sample comprising a test oligonucleotide. In certain embodiments, the test sample is a liquid laboratory sample (e.g., a buffer solution comprising a test oligonucleotide). In certain embodiments, the test sample is a biological sample obtained from a test subject, such as a mammal. As described herein, the term “biological fluid” refers to any bio-organic fluid produced by an organism and includes, but is not limited to, e.g., amniotic fluid, aqueous humour, vitreous humour, bile, blood or components of blood (e.g., serum or plasma), milk, cerebrospinal fluid (CSF), endolymph, perilymph, feces, lymph, mucus, pericardial fluid, peritoneal fluid, pleural fluid, pus, serous fluid, semen, sputum, synovial fluid, sweat, urine, saliva, tears, vaginal secretions and vomit. In certain embodiments, the biological fluid is blood or a blood component, such as serum. The methods of the invention may be used to directly analyze a biological fluid for the presence of a test oligonucleotide, such as a modified oligo, without processing the fluid or first isolating the oligonucleotide from the fluid. Accordingly, in certain embodiments, the test sample is an unprocessed biological fluid obtained from a mammal, such as a human.

As described herein, the test oligonucleotide (e.g., a modified oligo) may be a therapeutic oligonucleotide; such oligonucleotides often have been modified to be less susceptible to nucleases and more permeable to cells (e.g., vivo-morpholino oligos or oligos conjugated to amino acids). This increases their efficacy, but may present challenges for some traditional methods of measurement. For example, the use of PCR to detect/quantitate an oligonucleotide comprising a modified 3′ end may not be possible if elongation with PolyA is required. In contrast, the ELOSA methods described herein may be used to detect small oligonucleotides without the need for elongation. Additionally, oligonucleotide derivatization with morpholino groups and octaguanidine residues were shown to not affect the utility of the ELOSA methods described herein.

The test oligonucleotide may comprise deoxyribonucleotides and/or ribonucleotides in either single- or double-stranded form. If the test oligonucleotide is double-stranded, the sample should be treated to denature the strands prior to contact with the capture reagent. As discussed herein, the oligonucleotide may be modified to comprise one or more unnatural nucleic acids and/or the linkages between nucleotide bases may use alternative linking molecules.

Accordingly, in certain embodiments, the test oligonucleotide is a modified oligonucleotide (i.e., a modified oligo). In certain embodiments, the modified oligonucleotide comprises an unnatural nucleic acid(s) and/or backbone linkage modification(s). In certain embodiments, the modified oligonucleotide is a morpholino oligomer (i.e., a phosphorodiamidate morpholino oligomer (PMO)). As used herein, a morpholino oligomer has a backbone of methylenemorpholine rings and phosphorodiamidate linkages. In certain embodiments, the oligonucleotide has been modified to enhance cell penetration. In certain embodiments, the modified oligonucleotide is a vivo-morpholino. As used herein, a “vivo-morpholino” comprises a morpholino oligomer covalently linked to a delivery dendrimer (e.g., an octa-guanidine dendrimer).

The test oligonucleotide may be a small oligonucleotide that is shorter (e.g., an oligonucleotide therapeutic or miRNA), which often cannot be detected by traditional assay methods, such as standard polymerase chain reaction (PCR) without elongation or other modifications. For example, in certain embodiments, the test oligonucleotide is between about 10 to about 200 nucleotides in length (or any value in between). In certain embodiments, the oligonucleotide is about 10-150 nucleotides in length, about 10-100 nucleotides in length, about 10-90 nucleotides in length, about 10-80 nucleotides in length, about 10-70 nucleotides in length, about 10-60 nucleotides in length, about 10-50 nucleotides in length, about 10-40 nucleotides in length, about 15-40 nucleotides in length, about 20-40 nucleotides in length or about 25-35 nucleotides in length.

In certain embodiments, the test oligonucleotide is an antisense molecule. In certain embodiments, the test oligonucleotide is a splice modulating oligomer (SMO), a microRNA (miRNA), a siRNA, a sRNA, msRNA, ncRNA, tumor-derived DNA or a shRNA.

In certain embodiments, the test oligonucleotide is a vivo-morpholino PRLR SMO, e.g., a PRLR SMO as described herein, such as SEQ ID NO:4 or 7. In certain embodiments, the test oligonucleotide comprises a sequence having at least about 75%, 80%, 85%, 90%, 95%, 99% or 100% sequence identity to SEQ ID NO:4 or SEQ ID NO:7. In certain embodiments, the test oligonucleotide consists of a sequence having at least about 75%, 80%, 85%, 90%, 95%, 99% or 100% sequence identity to SEQ ID NO:4 or SEQ ID NO:7.

In certain embodiments, test oligonucleotide is a bacterial nucleic acid, such as bacterial DNA or RNA.

Capture Reagent

As described herein, the “capture reagent” refers to a compound that comprises a means to bind to a solid support (e.g., a linking group) and a means to bind to a test oligonucleotide and to a competitive oligonucleotide (e.g., a capture reagent oligonucleotide). Thus, the capture reagent is designed so that it binds to a solid support and is capable of separately binding to the test oligonucleotide and to the competition oligonucleotide (e.g., comprises an oligonucleotide sequence that is complementary to the two oligonucleotides). Accordingly, in certain embodiments, the capture reagent comprises an oligonucleotide (i.e., a capture reagent oligonucleotide) comprising a nucleic acid sequence that is complementary to the test oligonucleotide, as well as to the competition oligonucleotide. In certain embodiments, the capture reagent oligonucleotide comprises a sequence that is complementary to the test oligonucleotide (e.g., a modified oligo). In certain embodiments, the capture reagent oligonucleotide comprises a sequence that has at least about 80, 81, 82, 83, 84, 85, 86, 87, 88, 89, 90, 91, 92, 93, 94, 95, 96, 97, 98, 99 or 100% complementarity with the test oligonucleotide. In certain embodiments, the capture reagent oligonucleotide comprises a sequence that is complementary to the competition oligonucleotide. In certain embodiments, the capture reagent oligonucleotide comprises a sequence that has at least about 80, 81, 82, 83, 84, 85, 86, 87, 88, 89, 90, 91, 92, 93, 94, 95, 96, 97, 98, 99 or 100% complementarity with the competition oligonucleotide.

In certain embodiments, the capture reagent oligonucleotide is between about 10 to about 100 nucleotides in length (or any value in between). In certain embodiments, the oligonucleotide is about 10-90 nucleotides in length, about 10-80 nucleotides in length, about 10-70 nucleotides in length, about 10-60 nucleotides in length, about 10-50 nucleotides in length, about 10-40 nucleotides in length, about 15-40 nucleotides in length, about 20-40 nucleotides in length or about 25-35 nucleotides in length.

The capture reagent oligonucleotide may comprise deoxyribonucleotides and/or ribonucleotides. In certain embodiments, the capture reagent oligonucleotide is single stranded. As discussed herein, the oligonucleotide may be modified to comprise unnatural nucleic acids and/or the linkages between nucleotides may use alternative linking molecules.

As described herein, for use in the methods of the invention, the capture reagent is bound to a solid support (e.g., via a linking group).

As described herein, the “solid support” refers to any material capable of containing the reaction mixture. For example, the solid support may be a plate, such a multi-well plate, such as a 96 well plate (e.g., a DNA binding), a petri dish, a test tube, a cuvette, plates for fluorescence or luminescence, etc.

The nature of the linking group is not critical, and may be any group that can bind to a surface (i.e., of the solid support) using known chemistry, provided that it does not interfere with the binding between the capture reagent oligonucleotide and the test oligonucleotide/competition oligonucleotide. For example, in certain embodiments, the linking group may be an amide, amine (primary or secondary amine), carboxylic acid, alcohol or mercapto acid group.

In certain embodiments, the capture reagent further comprises a spacer group, wherein the spacer group joins the capture reagent oligonucleotide to the linking group. The nature of the spacer group is not critical, provided that it does not interfere with the binding between the capture reagent oligonucleotide and the test oligonucleotide or the competition oligonucleotide. The spacer group is typically a divalent organic radical having a molecular weight of from about 25 daltons to about 1000 daltons, or from about 25 daltons to about 500 daltons, or from about 25 daltons to about 300 daltons.

The spacer group typically has a length of from about 5 angstroms to about 100 angstroms using standard bond lengths and angles. More specifically, the spacer group has a length of from about 10 angstroms to about 50 angstroms. In certain embodiments, the spacer group separates the capture reagent oligonucleotide from the linking group by about 5 angstroms to about 40 angstroms, inclusive, in length.

In another embodiment of the invention the spacer group is a divalent, branched or unbranched, saturated or unsaturated, hydrocarbon chain, having from 2 to 20 carbon atoms, wherein one or more (e.g. 1, 2, 3, or 4) of the carbon atoms is optionally replaced by (—O—) or (—N—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the spacer group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N—), and wherein the chain is optionally substituted on carbon with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In another embodiment of the invention the spacer group is a divalent, branched or unbranched, saturated or unsaturated, hydrocarbon chain, having from 2 to 15 carbon atoms, wherein one or more (e.g. 1, 2, 3, or 4) of the carbon atoms is optionally replaced by (—O—) or (—N—).

In another embodiment of the invention the spacer group is a divalent, branched or unbranched, saturated or unsaturated, hydrocarbon chain, having from 8 to 15 carbon atoms.

In another embodiment of the invention the spacer group is a divalent, branched or unbranched, saturated hydrocarbon chain, having from 8 to 15 carbon atoms.

In another embodiment of the invention the spacer group is a divalent, unbranched, saturated hydrocarbon chain, having from 8 to 15 carbon atoms.

In another embodiment of the invention the spacer group is a divalent, unbranched, saturated hydrocarbon chain, having 12 carbon atoms.

In another embodiment of the invention, the spacer group is a 12 carbon methylene chain.

In another embodiment of the invention the spacer group is a divalent radical formed from a protein.

In another embodiment of the invention the spacer group is a divalent radical formed from a peptide.

In another embodiment of the invention the spacer group is a divalent radical formed from an amino acid.

In certain embodiments, the capture reagent comprises, in order: a capture reagent oligonucleotide, a spacer group and a linker group. The capture reagent oligonucleotide may be in either the 5′ to 3′ orientation or 3′ to 5′ orientation. Accordingly, in certain embodiments, the capture reagent comprises a compound of formula (I):

A-B-C (I)
wherein:
   A is a capture reagent oligonucleotide as described herein (e.g., SEQ ID NO:5 or SEQ ID NO:8);
   B is a spacer group as described herein (e.g., a 12 carbon methylene chain); and
   C is a linking group as described herein (e.g., an amine group).

In certain embodiments, the capture reagent consists of a compound of formula (I).

Competition Oligonucleotide

The “competition oligonucleotide” refers to an oligonucleotide that is capable of competing with the test oligonucleotide for binding to the capture reagent. Accordingly, the competition oligonucleotide comprises a sequence that is complementary to the capture reagent oligonucleotide and is capable of specifically binding to the capture reagent oligonucleotide. In certain embodiments, the competition oligonucleotide comprises a sequence that has at least about 80, 81, 82, 83, 84, 85, 86, 87, 88, 89, 90, 91, 92, 93, 94, 95, 96, 97, 98, 99 or 100% complementarity with the capture reagent oligonucleotide. In certain embodiments, the competition oligonucleotide comprises a sequence that has at least about 80, 81, 82, 83, 84, 85, 86, 87, 88, 89, 90, 91, 92, 93, 94, 95, 96, 97, 98, 99 or 100% sequence identity to the test oligonucleotide. In certain embodiments, the competition oligonucleotide and the test oligonucleotide have 100% sequence identity.

In certain embodiments, the competition oligonucleotide is between about 10 to about 100 nucleotides in length (or any value in between). In certain embodiments, the oligonucleotide is about 10-90 nucleotides in length, about 10-80 nucleotides in length, about 10-70 nucleotides in length, about 10-60 nucleotides in length, about 10-50 nucleotides in length, about 10-40 nucleotides in length, about 15-40 nucleotides in length, about 20-40 nucleotides in length or about 25-35 nucleotides in length.

The competition oligonucleotide may comprise deoxyribonucleotides and/or ribonucleotides. In certain embodiments, the competition oligonucleotide is single stranded. The competition oligonucleotide may be modified to comprise unnatural nucleic acids and/or the linkages between nucleotides may use alternative linking molecules. In certain embodiments, the test oligonucleotide is modified (e.g., comprises unnatural nucleic acids and/or backbone modifications) and the competition oligonucleotide is not modified.

As described herein, the competition oligonucleotide is operably linked to an enzyme (e.g., at the 5′ or 3′ end of the oligonucleotide; e.g., at the 5′ end of the oligonucleotide). Chemistries that can be used to link the enzyme to the oligonucleotide are known in the art. Any synthetically feasible point on the competition oligonucleotide and on the enzyme (i.e., any functional group) may be used to operably link the two components, provided the linkage does not interfere with the oligonucleotide binding to the capture reagent or with the activity of the enzyme. In certain embodiments, the competition oligonucleotide is operably linked to the enzyme by means of a covalent bond. For example, the enzyme may be operably linked to the competition oligonucleotide through an ether, ester, amide, amine or sulfur bond.

Enzyme—Substrate

As described herein, the enzyme operably linked to the competition oligonucleotide may be any enzyme that is capable of specifically binding to a particular substrate and catalyzing a chemical reaction to generate a reaction product, wherein the reaction product is capable of being detected and/or quantified. In certain embodiments, the reaction product is detected and/or quantified spectrophotometrically or fluorometrically (i.e., the enzyme catalyzes the conversion of a chromogenic substrate into a colored reaction product or the reaction product emits light at a particular wavelength). Enzyme-substrate pairs, which generate such reaction products, as well as methods for detecting such products, are known in the art.

In certain embodiments, the substrate is a chromogenic substrate, wherein the reaction product can be detected spectrophotometrically.

In certain embodiments, the substrate is a dye or fluorophore, wherein the reaction product can be detected fluorometrically.

In certain embodiments, the enzyme is horseradish peroxidase (HRP) and the substrate is a horseradish peroxidase (HRP) substrate. In certain embodiments, the horseradish peroxidase (HRP) substrate is selected from the group consisting of:

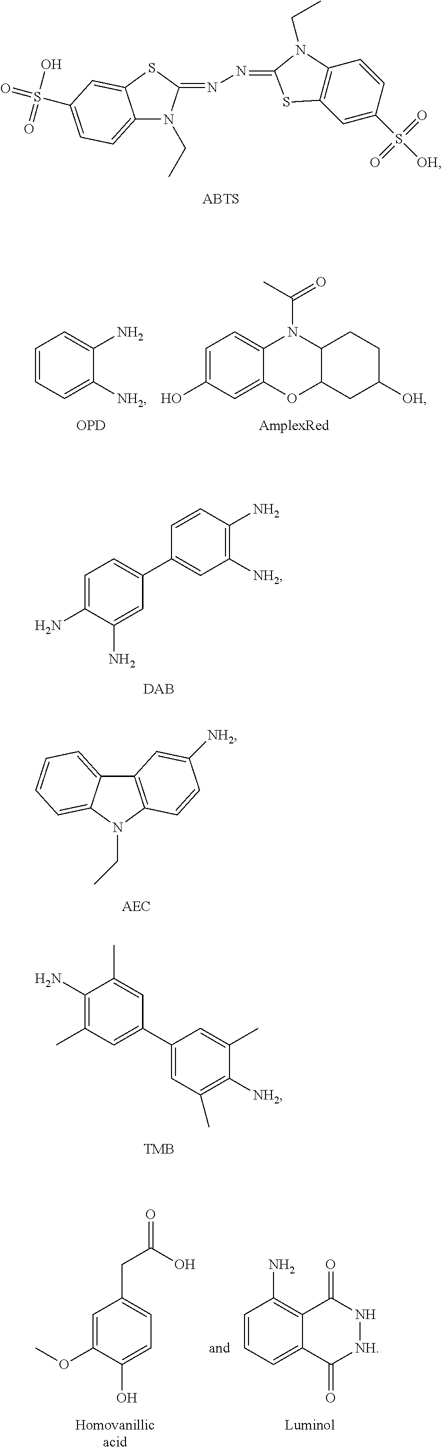

In certain embodiments, the enzyme is alkaline phosphatase (AP) and the substrate is an AP substrate, such as PNPP (p-nitrophenyl phosphate, disodium salt).

In certain embodiments, the enzyme is beta-galactosidase (β-gal) and the substrate is a β-gal substrate, such as o-nitrophenyl-β-D-galactopyranoside (ONPG), naphthol-AS-Bl-β-D-galactopyranoside (Nap-GAL), or 4 methyl-umbelliferyl-β-D-galactopyranoside (MUm-gal).

As described herein, a secondary agent may be needed to catalyze the reaction between the enzyme and the substrate. The “secondary agent” may be any compound capable of catalyzing a reaction between the enzyme and substrate. For example, if the enzyme is HRP, hydrogen peroxide may need to be added to catalyze the reaction between HRP and the HRP substrate. Accordingly, in certain embodiments, the methods further comprise contacting the reaction mixture and substrate with a secondary agent to generate the enzyme-substrate reaction product.

Reaction Mixture

To ensure adequate competition between the test oligonucleotide and the competition oligonucleotide, a limiting amount of the capture reagent should be used. In certain embodiments, less than about 3.0 picomoles of the capture reagent are used. In certain embodiments, less than about 2.5 picomoles of the capture reagent are used. In certain embodiments, about 2.0 picomoles of the capture reagent are used.

In certain embodiments, about 0.05 to about 1.5 pmoles of the competition oligonucleotide are used. In certain embodiments, about 0.5 to about 1.0 pmoles of the competition oligonucleotide are used. In certain embodiments, about 0.5 picomoles of the competition oligonucleotide are used.

The reaction mixture will ideally constitute a relatively small volume, for example about 50 μl to about 200 μl, although greater or lesser volumes may be employed. Small volumes allow components of the invention to be conserved, which reduces the cost of the assay (e.g., the capture reagent, competition oligonucleotide, substrate, etc.).

In certain embodiments, a total final volume of 100 μl (e.g., in 0.05M TrisHCl (pH 7.5)) is used: about 0.2 to about 1 picomole of the competition oligonucleotide operably linked to the enzyme; and about 0.01 to 2.5 pmoles standard oligonucleotide (or modified oligo) or test sample (e.g., 10-25 μl serum or plasma comprising the test oligonucleotide (e.g., a modified oligo)).

Incubation

Prior to contact with the test sample, the capture reagent is contacted with the solid support under conditions suitable for binding between the two elements to occur. Specifically, the capture reagent is incubated with the solid support for a time sufficient to allow maximal binding to the support, and varies with the conditions, the type of solid support, and the length of the oligonucleotide. In certain embodiments, the capture reagent and the solid support are incubated together for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47 or 48 hours. In certain embodiments, the capture reagent and the solid support are incubated in the presence of a buffer solution. In certain embodiments, the capture reagent and the solid support are incubated at a temperature between about 1° C. to about 10° C., or between about 2° C. to about 7° C. or at about 4° C. For example, in certain embodiments, the capture reagent (e.g., a capture reagent comprising oligonucleotide 29 nucleotides in length+12-(CH₂) spacer+linking group) is incubated with a 96-well, DNA-binding plate for >24 h in 0.05M Na phosphate buffer (pH 8.5)—1 mM EDTA at 4° C.

As described herein, the methods of the invention comprise contacting the test sample, which comprises the test oligonucleotide with i) the capture reagent bound to a solid support; and ii) the competition oligonucleotide operably linked to an enzyme, to thereby create a reaction mixture. The reaction mixture should be incubated under conditions suitable for hybridization to occur between the test oligonucleotide and the capture reagent and/or between the competition oligonucleotide and the capture reagent. Incubation time and conditions can vary from a few minutes to 24 hours or longer depending upon the sensitivity required. In certain embodiments, the reaction mixture is incubated for at least about 5, 10, 15, 20, 25, 30, 35, 40, 45, 50, 55 or 60 minutes. In certain embodiments, the reaction mixture is incubated for at least about 2, 3, 4, 5, 6, 7, 8, 9, 10, 11, 12, 13, 14, 15, 16, 17, 18, 19, 20, 21, 22, 23 or 24 hours, or more. In certain embodiments, the reaction mixture is incubated for about 15 hrs. Incubation temperatures can generally vary from about 4° C. to about 100° C. or higher. In certain embodiments, the incubation temperature is about 22° C. to about 65° C. In certain embodiments, the incubation temperature is about 22° C. to about 37° C. In certain embodiments, the incubation temperature is about 37° C.

As described herein, the methods of the invention also comprise contacting the reaction mixture with a substrate that specifically binds to the enzyme. The reaction mixture and the substrate should be contacted under conditions suitable for catalyzing a chemical reaction between the enzyme and substrate (i.e., to generate the enzyme-substrate reaction product). The amount of substrate to be added and reaction conditions will be specific to the substrate-enzyme combination selected and can be determined/optimized by one skilled in the art.

Measurement of the Enzyme-Substrate Reaction Product

The amount of the competition oligonucleotide bound to the capture reagent may be measured using a substrate recognized by the enzyme. As discussed above, the amount of substrate to be added and reaction conditions will be specific to the substrate-enzyme combination selected and can be determined/optimized by one skilled in the art.

As the test oligonucleotide (e.g., a modified oligo) and the competition oligonucleotide compete for binding to the capture oligonucleotide, the concentration of the enzyme-substrate reaction product will be inversely proportional to the concentration of the test oligonucleotide (i.e., the greater the concentration of the test oligonucleotide (e.g., a modified oligo) in the sample, the less enzyme-substrate reaction product will be generated). As described herein, the enzyme-substrate reaction product may be measured using techniques known in the art. For example, in certain embodiments, the substrate may be a chromogenic substrate, which changes color upon being acted on by the enzyme. In other embodiments, the substrate may be a dye or fluorophore that changes absorbance upon being acted upon by the enzyme. Accordingly, it may be possible to detect the presence of the test oligonucleotide with unassisted visual inspection of the test reaction mixture. However, the concentration of the enzyme-substrate reaction products in the test and control mixtures may also be measured spectrophotometrically using a spectrophotometer or fluorometrically using a fluorometer, or any other devices capable of detecting absorbance/fluorescent light emission in a quantitative or qualitative fashion.

Vessel for Steps of the Invention—Solid Support

The steps of the invention (e.g., contacting, incubating, measuring, etc.) may be performed in a single vessel (i.e., the solid support). For example, as described herein, the capture reagent is bound to a solid support, which may be, e.g., a plate, such a multi-well plate, such as a 96 well plate (e.g., a DNA binding), a petri dish, a test tube (e.g., a microfuge tube), a cuvette, plates for fluorescence or luminescence etc. Accordingly, the solid support may be used as a vessel for performing the steps of the invention.

Administration

As described herein, methods of the invention may further comprise administering a therapeutic agent to a mammal (e.g., a mammal diagnosed with a particular disease, disorder or condition using a method described herein). Such a therapeutic agent may be formulated as pharmaceutical composition and administered to a mammalian host, such as a human patient in a variety of forms adapted to the chosen route of administration, i.e., orally or parenterally, by intravenous, intramuscular, topical or subcutaneous routes.

Thus, the therapeutic agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver a therapeutic agent to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of therapeutic agents can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic agent, or an active salt or derivative thereof, required for use in treatment will vary not only with the particular salt selected but also with the route of administration, the nature of the condition being treated and the age and condition of the patient and will be ultimately at the discretion of the attendant physician or clinician.

The therapeutic agent is conveniently formulated in unit dosage form. In one embodiment, the invention provides a composition comprising a therapeutic agent formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Compounds and Compositions

The present invention further provides compounds and compositions as described herein, which may be used for practicing the present methods.

Accordingly, certain embodiments of the invention provide a capture reagent as described herein. In certain embodiments, the capture reagent comprises a capture reagent oligonucleotide that is between about 10 to about 100 nucleotides in length (or any value in between). In certain embodiments, the oligonucleotide is about 10-90 nucleotides in length, about 10-80 nucleotides in length, about 10-70 nucleotides in length, about 10-60 nucleotides in length, about 10-50 nucleotides in length, about 10-40 nucleotides in length, about 15-40 nucleotides in length, about 20-40 nucleotides in length or about 25-35 nucleotides in length. In certain embodiments, the capture reagent comprises a capture reagent oligonucleotide comprising a sequence that specifically binds to a PRLR SMO (e.g., AAAAAGCCCTTCTATTGAAACACAGATACAAAA (SEQ ID NO:4) or AAAAAGCCCTTCTATTAAAACACAGACACAAAA (SEQ ID NO:7)). In certain embodiments, the capture reagent comprises a capture reagent oligonucleotide comprising a sequence having at least about 75%, 80%, 85%, 90%, 95%, 99% or 100% sequence identity to TTTTACTCCGATCGAATCTCGTCTTTTTT (SEQ ID NO:2), TTTTGTATCTGTGTTTCAATAGAAGGGCTTTTT (SEQ ID NO:5) or TTTTGTGTCTGTGTTTTAATAGAAGGGCTTTTT (SEQ ID NO:8). In certain embodiments, the capture reagent comprises a capture reagent oligonucleotide consisting of a sequence having at least about 75%, 80%, 85%, 90%, 95%, 99% or 100% sequence identity to SEQ ID NO:2, SEQ ID NO:5 or SEQ ID NO:8. In certain embodiments, the capture reagent comprises a compound of formula (I), wherein A comprises a sequence having at least about 75%, 80%, 85%, 90%, 95%, 99% or 100% sequence identity to SEQ ID NO:2, SEQ ID NO:5 or SEQ ID NO:8; B is a spacer group described herein, and C is a linking group described herein. In certain embodiments, the capture reagent comprises a compound of formula (I), wherein A comprises a sequence having at least about 75%, 80%, 85%, 90%, 95%, 99% or 100% sequence identity to SEQ ID NO:2, SEQ ID NO:5 or SEQ ID NO:8; B is a 12 carbon methylene chain, and C is an amine group. In certain embodiments, the capture reagent is bound to a solid support (e.g., a solid support described herein, such as a plate, such a multi-well plate, such as a 96 well plate (e.g., a DNA binding), a petri dish, a test tube (e.g., a microfuge tube), a cuvette, plates for fluorescence or luminescence etc.).

Certain embodiments of the invention provide a competition oligonucleotide as described herein. In certain embodiments, the competition oligonucleotide is between about 10 to about 100 nucleotides in length (or any value in between). In certain embodiments, the oligonucleotide is about 10-90 nucleotides in length, about 10-80 nucleotides in length, about 10-70 nucleotides in length, about 10-60 nucleotides in length, about 10-50 nucleotides in length, about 10-40 nucleotides in length, about 15-40 nucleotides in length, about 20-40 nucleotides in length or about 25-35 nucleotides in length. Certain embodiments of the invention provide a competition oligonucleotide comprising a sequence having at least about 75%, 80%, 85%, 90%, 95%, 99% or 100% sequence identity to AAAAAAGACGAGATTCGATCGGAGTAAAA (SEQ ID NO:3), AAAAAGCCCTTCTATTGAAACACAGATACAAAA (SEQ ID NO:6) or AAAAAGCCCTTCTATTAAAACACAGACACAAAA (SEQ ID NO:9). In certain embodiments, competition oligonucleotide consists of a sequence having at least about 75%, 80%, 85%, 90%, 95%, 99% or 100% sequence identity to SEQ ID NO:3, SEQ ID NO:6 or SEQ ID NO:9. In certain embodiments, the competition oligonucleotide is operably linked to an enzyme (e.g., an enzyme described herein). In certain embodiments, the competition oligonucleotide is operably linked to horseradish peroxidase. In certain embodiments, the competition oligonucleotide is operably linked to alkaline phosphatase (AP). In certain embodiments, the competition oligonucleotide is operably linked to beta-galactosidase (β-gal).

### Kits

The present invention further provides kits for practicing the present methods. Accordingly, certain embodiments of the invention provide a kit for detecting and/or quantifying a test oligonucleotide (e.g., a modified oligonucleotide, such as a phosphorodiamidate morpholino) in a test sample, such as a biological fluid, wherein the kit comprises a capture reagent as described herein, standard test oligonucleotides (e.g., standard modified oligos) for calibration, a competition oligonucleotide operably linked to an enzyme as described herein, an enzyme specific-substrate as described herein and instructions for use. Such kits may optionally contain one or more of: a positive and/or negative control, RNase-free water, and one or more buffers. In certain embodiments, a kit may further include RNase-free laboratory plasticware (e.g., a plate(s), such a multi-well plate(s), such as a 96 well plate(s) (e.g., DNA binding), a petri dish(es), a test tube(s), a cuvette(s), a plate(s) for fluorescence or luminescence etc.). For example, in certain embodiments, the kit further comprises a solid support. In certain embodiments, the capture reagent is bound to a solid support. In certain embodiments, kit comprises the instructions for binding the capture reagent to a solid support.

### Certain Definitions

As used herein, the term “about” means±10%.

“Operably-linked” refers to the association two chemical moieties so that the function of one is affected by the other, e.g., an arrangement of elements wherein the components so described are configured so as to perform their usual function.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The terms “nucleotide sequence” and “nucleic acid sequence” refer to a sequence of bases (purines and/or pyrimidines) in a polymer of DNA or RNA, which can be single-stranded or double-stranded, optionally containing synthetic, non-natural or altered nucleotide bases capable of incorporation into DNA or RNA polymers, and/or backbone modifications (e.g., a modified oligomer, such as a morpholino oligomer, phosphorodiamate morpholino oligomer or vivo-mopholino). The terms “oligo”, “oligonucleotide” and “oligomer” may be used interchangeably and refer to such sequences of purines and/or pyrimidines. The terms “modified oligos”, “modified oligonucleotides” or “modified oligomers” may be similarly used interchangeably, and refer to such sequences that contain synthetic, non-natural or altered bases and/or backbone modifications (e.g., chemical modifications to the internucleotide phosphate linkages and/or to the backbone sugar). Additionally, a modified oligonucleotide may be covalently linked to a delivery molecule (e.g., a dendrimer, e.g., an octa-guanidine dendrimer). Accordingly, the term modified oligonucleotide includes morpholino oligonucleotides, such as vivo-morpholinos.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methyl cytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propyl cytosine; 5-ethyluracil; 5-ethylcytosine; 5-butyluracil; 5-pentyluracil; 5-pentylcytosine; and 2,6-diaminopurine; methylpsuedouracil; 1-methylguanine; 1-methylcytosine. Backbone modifications are similarly known in the art, and include, chemical modifications to the phosphate linkage (e.g., phosphorodiamidate, phosphorothioate (PS), N3′phosphoramidate (NP), boranophosphate, 2′,5′phosphodiester, amide-linked, phosphonoacetate (PACE), morpholino, peptide nucleic acid (PNA) and inverted linkages (5′-5′ and 3′-3′ linkages)) and sugar modifications (e.g., 2′-O-Me, UNA, LNA).

The oligonucleotides described herein may be synthesized using standard solid or solution phase synthesis techniques which are known in the art. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The following terms are used to describe the sequence relationships between two or more nucleotide sequences: (a) “reference sequence,” (b) “comparison window,” (c) “sequence identity” (d) “percentage of sequence identity,” (e) “substantial identity” and (f) “complementarity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity, including sequence complementarity, between any two sequences can be accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, J M B, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity or complementarity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

(f) The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T (A:U for RNA) and G:C nucleotide pairs).

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine. The term “peptide” describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula I through the carboxy terminus, the amino terminus, or through any other convenient point of attachment, such as, for example, through the sulfur of a cysteine. Preferably a peptide comprises 3 to 25, or 5 to 21 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

The term “mammal” as used herein refers to humans, higher non-human primates, rodents, domestic, cows, horses, pigs, sheep, dogs and cats. In one embodiment, the mammal is a human.

The invention will now be illustrated by the following non-limiting Examples.

### Example 1

General methods for the ELSOA are shown below; however, conditions may vary depending on the oligonucleotide to be tested. For example, capture binding, hybridization and incubation times may vary, as well, as the buffers and concentrations thereof. The specific ELOSA conditions for a given test oligonucleotide (e.g., a modified oligo) may be optimized by one skilled in the art.

Binding of the Capture Reagent.

The capture reagent was synthesized using a sequence antisense to the test oligonucleotide, along with an amino-terminus and a 12 carbon aliphatic spacer. The amino-terminus allows binding to the wells of a 96-well, polystyrene DNA-binding plate and the spacer minimizes steric hindrance during hybridization of the test oligonucleotides. The capture reagent (2 picomoles/well) was incubated in 100 μl 0.05M phosphate (pH 8.5) —1 mM EDTA at 4° C. for 24 h or longer.

If the capture reagent was 2 pmoles with the HRP-PRLR SMO in the 0.5 pmole range in the presence of serum, competition with Antimaia was easily detected above 0.2 pmoles.

Blocking.

After washing (3 times with 0.05M TrisHCl (pH 8)—1 mM EDTA), the plate was blocked to reduce non-specific binding (200 μl 3% bovine serum albumin—0.05M TrisHCl (pH 8)—1 mM EDTA at 37° C. for 4 h).

Hybridization.

After washing 3 times, the test oligo and a constant concentration of horseradish peroxidase-labeled oligonucleotide (i.e., the competition oligonucleotide with a sequence the same as test oligo) was incubated with the capture reagent. The amount of competition oligonucleotide hybridized to the capture reagent is inversely proportional to the total amount of test oligonucleotide present. Incubations were in a total final volume of 100 μl in 0.05M TrisHCl (pH 7.5)—1 mM EDTA, with Test oligo (variable doses up to 1 pmole), constant HRP-Oligo (0.5-1 pmole), and a total serum volume of 10 to 25 μl. Incubations were at 37° C. for 15 h.

Measurement of HRP-Oligonucleotide Bound.

After washing 3 times with TBST, the competition oligonucleotide bound to the capture reagent was measured using 100 μl 0.1 mg/ml TMB—1% hydrogen peroxide. After color development, the reaction was stopped with 50 μl 1N H₂SO₄, and the amount of colored product was measured spectrophotometrically at A₄₅₀.

### Example 2

### ELOSA Method for Measuring a Splice-Modulating Oligomer (SMO) Affecting the Prolactin Receptor (PRLR) or a Nonsense Control SMO

Applicability of the ELOSA method was tested using a vivo-morpholino SMO for the prolactin receptor (PRLR), which has both a phosphorodiamidate morpholino backbone and octaguanidine derivatizations. Treatment with this PRLR SMO specifically results in a loss of the growth-promoting PRLR without loss of the growth-inhibiting splice form of the PRLR. The PRLR SMO is well-tolerated and results in an 80% reduction in metastatic spread in two orthotopic models of breast cancer (Yonezawa et al., Cancer Lett. 2015 Sep. 28; 366(1):84-92). The PRLR SMO is described in U.S. Patent Publication No. 2015-0337310, which is incorporated by reference herein. As this SMO is currently being tested as a cancer therapeutic, the ability to accurately and efficiently detect and quantify delivery and pharmacokinetics is important for drug development and clinical use. The results described below demonstrate that ELOSA can effectively detect small quantities of the PRLR SMO and control SMO in serum.

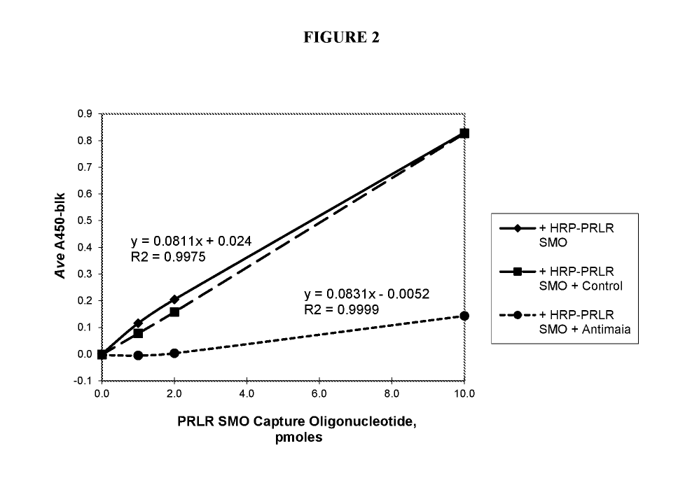

As shown in Table 1 below, the following oligos were synthesized for both mouse and human assays: vivo-morpholinos PRLR SMO and Control SMO (SMO of no predicted biological activity, established via BLAST analysis) (Gene Tools, Philomath, Oreg.); Capture PRLR SMO oligo (antisense to PRLR SMO with an amino-12C link, not a vivo-morpholino), Capture Control SMO oligo (antisense to control SMO with an amino-12C link, not a vivo-morpholino), HRP-PRLR SMO (same sequence as PRLR SMO, not a vivo-morpholino), and HRP-Control SMO (same sequence as control SMO, not a vivo-morpholino) (Biosynthesis, Lewisville, Tex.). For clarity, the PRLR SMO vivo-morpholino is referred to as Antimaia to distinguish it from the oligonucleotides being used for competition or capture, which are called HRP-PRLR SMO and capture PRLR SMO, respectively. The applicability of the ELOSA method is demonstrated by the experiments shown in FIGS. 2-4, which utilized the mouse sequences. The methods used for these experiments are described in Example 1. Similar experiments may be performed using the human sequences, which are also shown in Table 1.

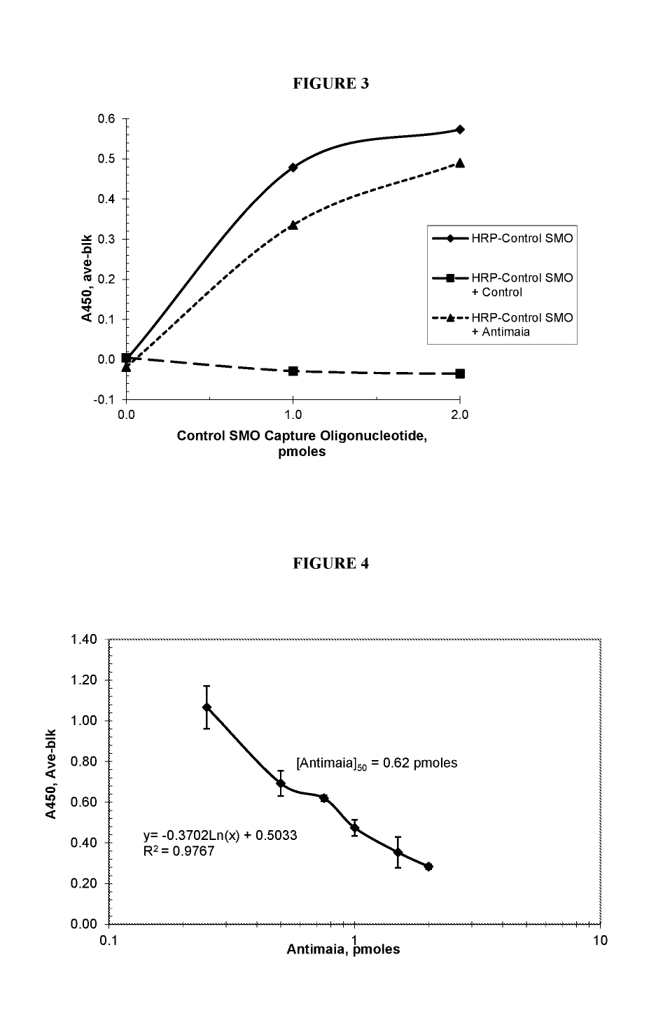

As shown in FIG. 2, ELOSA is specific. The control vivo-morpholino SMO did not hybridize to the Capture PRLR SMO or compete with Antimaia for displacement of HRP-PRLR SMO. Additionally, the assay was determined to be applicable to other oligonucleotides. As shown in FIG. 3, the control vivo-morpholino SMO (Control; 5 pmoles) competed with HRP-Control SMO (1 pmole) for binding to the Capture Control SMO (5 pmoles), whereas Antimaia (5 pmoles) had essentially no effect. As illustrated in FIG. 4, the assay was also shown to be dose dependent and functional at very low concentrations. Specifically, it was found that increased absorbance may be obtained in the presence of serum and the sensitivity of the assay was between about 0.25 and about 2 pmoles (Capture PRLR-SMO=2 pmoles; HRP-PRLR-SMO=0.5 pmole). The presence of serum did not interfere with the assay; in fact, enhanced absorbance was observed without loss of sensitivity. When Antimaia was tested in serum samples, the sample is also assayed using a Control SMO Capture Oligonucleotide; this served as a control for any potential nonspecific absorbance.

TABLE 1
CONTROL SEQUENCES

| | Oligonucleotide | |
|---|---|---|
| Reagent Type | Name | Sequence |
| Test | Control SMO | 5′-AAAAAAGACGAGATTCGATCGGAGTAAAA-3′ |
| Oligonucleotide | (vivo-morpholino) | (SEQ ID NO: 1) |
| Capture Reagent | Capture Control | [Amino-12 carbon methylene chain]-TTTTACTCCGATCGA |
| | SMO oligo | ATCTCGTCTTTTTT (SEQ ID NO: 2) |
| Competition | HRP-Control SMO | HRP-5′-AAAAAAGACGAGATTCGATCGGAGTAAAA-3′ |
| Oligonucleotide | | (SEQ ID NO: 3) |
| Operably linked | | |
| to an Enzyme | | |

MOUSE SEQUENCES

| | Oligonucleotide | |
|---|---|---|
| Reagent Type | Name | Sequence |
| Test | PRLR SMO | 5′-AAAAAGCCCTTCTATTGAAACACAGATACAAAA-3′ |
| Oligonucleotide | (i.e., Antimaia; | (SEQ ID NO: 4) |
| | vivo-morpholino) | |
| Capture Reagent | Capture PRLR | [Amino-12 carbon methylene chain]-TTTTGTATCTGTGTTT |
| | SMO oligo | CAATAGAAGGGCTTTTT (SEQ ID NO: 5) |
| Competition | HRP-PRLR SMO | HRP-5′-AAAAAGCCCTTCTATTGAAACACAGATACA |
| Oligonucleotide | | AAA-3′ (SEQ ID NO: 6) |
| Operably linked | | |
| to an Enzyme | | |

HUMAN SEQUENCES

| | Oligonucleotide | |
|---|---|---|
| Reagent Type | Name | Sequence |
| Test | PRLR SMO | 5′-AAAAAGCCCTTCTATTAAAACACAGACACAAAA-3′ |
| Oligonucleotide | (i.e., Antimaia; | (SEQ ID NO: 7) |
| | vivo-morpholino) | |
| Capture Reagent | Capture PRLR | [Amino-12 carbon methylene chain]-TTTTGTGTCTGTGTT |
| | SMO oligo | TTAATAGAAGGGCTTTTT (SEQ ID NO: 8) |
| Competition | HRP-PRLR SMO | HRP-5′-AAAAAGCCCTTCTATTAAAACACAGACACA |
| Oligonucleotide | | AAA-3′ (SEQ ID NO: 9) |
| Operably linked | | |
| to an Enzyme | | |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for the detection and/or quantification of a test oligonucleotide in a test sample comprising:
a) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture;
b) contacting the reaction mixture with a substrate that specifically binds to the enzyme, thereby generating an enzyme-substrate reaction product; and
c) measuring the concentration of the enzyme-substrate reaction product, so as to detect and/or quantify the test oligonucleotide.

2. A method for the detection and/or quantification of a test oligonucleotide in a test sample comprising:
a) contacting the test sample with i) a capture reagent bound to a first solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a test reaction mixture;
b) contacting the test reaction mixture with a substrate that specifically binds to the enzyme, thereby generating a test enzyme-substrate reaction product;
c) measuring the concentration of the test enzyme-substrate reaction product;
d) contacting a control sample comprising a predetermined amount of the test oligonucleotide with i) a capture reagent bound to a second solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a control reaction mixture;
e) contacting the control reaction mixture with a substrate that specifically binds to the enzyme, thereby generating a control enzyme-substrate reaction product; and
f) measuring the concentration of the control enzyme-substrate reaction product, wherein the relative concentration of the test enzyme-substrate reaction product to the control enzyme-substrate reaction product indicates the presence and/or quantity of the test oligonucleotide.

3. The method of claim 2, wherein the predetermined amount of the test oligonucleotide is no test oligonucleotide, such that a concentration of the test enzyme-substrate reaction product less than the concentration of the control enzyme-substrate reaction product indicates that the test sample comprises the test oligonucleotide.

4. A method for diagnosing a disease, disorder or condition in a mammal comprising:
a) detecting the presence and/or concentration of a test oligonucleotide in a test sample obtained from the mammal by:
1) contacting the test sample with i) a capture reagent bound to a solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a reaction mixture;
2) contacting the reaction mixture with a substrate that specifically binds to the enzyme, thereby generating an enzyme-substrate reaction product; and
3) measuring the concentration of the enzyme-substrate reaction product, so as to detect and/or quantify the test oligonucleotide;
b) diagnosing the mammal with the disease, disorder or condition when the presence or certain concentration of the test oligonucleotide is detected.

5. The method of claim 4, further comprising administering a therapeutic agent to the diagnosed mammal.

6. The method of claim 4, wherein the disease, disorder or condition is cancer or a bacterial infection.

7. A method for evaluating the effectiveness of therapeutic agent in a mammal comprising:
a) detecting the presence and/or concentration of a test oligonucleotide in a first test sample from the mammal by:
1) contacting the first test sample with i) a capture reagent bound to a first solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a first reaction mixture;
2) contacting the first reaction mixture with a substrate that specifically binds to the enzyme, thereby generating a first enzyme-substrate reaction product; and
3) measuring the concentration of the first enzyme-substrate reaction product, so as to detect and/or quantify the test oligonucleotide in the first sample;
b) administering a therapeutic agent to the mammal;
c) detecting the presence and/or concentration of the test oligonucleotide in a subsequent second test sample from the mammal by:
1) contacting the second test sample with i) a capture reagent bound to a second solid support, wherein the capture reagent comprises an oligonucleotide comprising a nucleic acid sequence complementary to the test oligonucleotide; and ii) a competition oligonucleotide operably linked to an enzyme, wherein the competition oligonucleotide comprises a nucleic acid sequence complementary to the capture oligonucleotide; thereby creating a second reaction mixture;
2) contacting the second reaction mixture with a substrate that specifically binds to the enzyme, thereby generating a second enzyme-substrate reaction product; and
3) measuring the concentration of the second enzyme-substrate reaction product, so as to detect and/or quantify the test oligonucleotide in the second test sample;
d) determining the effectiveness of the therapeutic agent by comparing the presence/concentration of the test oligonucleotide in the first test sample to the presence/concentration of the test oligonucleotide in the second test sample.

8. The method of any one of claims 1-7, further comprising incubating the reaction mixture(s) under conditions suitable for hybridization to occur between the test oligonucleotide and the capture reagent and/or between the competition oligonucleotide and the capture reagent.

9. The method of any one of claims 1-7, further comprising washing the reaction mixture(s) one or more times with a buffer prior to contacting the reaction mixture with a substrate.

10. The method of any one of claims 1-7, further comprising contacting the capture reagent bound to the solid support with a blocking solution that reduces non-specific binding, prior to contact with the test sample.

11. The method of any one of claims 1-7, further comprising washing the capture reagent bound to the solid support one or more times with a buffer solution, prior to contact with the test sample.

12. The method of any one of claims 1-7, wherein the test sample is a biological fluid.

13. The method of claim 12, wherein the biological fluid is selected from blood, serum, milk, cerebrospinal fluid, urine, saliva and tears.

14. The method of claim 13, wherein the biological fluid is serum.

15. The method of any one of claims 1-7, wherein the test oligonucleotide is about 15 to about 40 nucleotides in length.

16. The method of any one of claims 1-7, wherein the test oligonucleotide is a modified oligonucleotide comprising one or more unnatural nucleic acid(s) and/or backbone linkage modification(s).

17. The method of claim 16, wherein the modified oligonucleotide is a vivo-morpholino.

18. The method of any one of claims 1-7, wherein the test oligonucleotide is an anti-sense molecule.

19. The method of any one of claims 1-7, wherein the test oligonucleotide is selected from the group consisting of a bacterial nucleic acid, a splice modulating oligomer (SMO), a microRNA (miRNA), a siRNA, a sRNA, a msRNA, a ncRNA, tumor-derived DNA and a shRNA.

20. The method of any one of claims 1-7, wherein the capture reagent comprises an oligonucleotide comprising a nucleic acid sequence that has at least about 85% complementarity to the test oligonucleotide.

21. The method of any one of claims 1-7, wherein the capture reagent comprises an oligonucleotide comprising a nucleic acid sequence that has at least about 95% complementarity to the test oligonucleotide.

22. The method of any one of claims 1-7, wherein capture reagent oligonucleotide is about 15 to about 40 nucleotides in length.

23. The method of any one of claims 1-7, wherein the solid support is a DNA binding plate.

24. The method of any one of claims 1-7, wherein the capture reagent is bound to the solid support via a linking group.

25. The method of any one of claim 24, wherein the linking group is an amide, amine, carboxylic acid, alcohol or mercapto acid group.

26. The method of any one of claims 1-7, wherein the capture reagent further comprises a spacer group, wherein the spacer group joins the capture reagent oligonucleotide to the linking group.

27. The method of claim 26, wherein the spacer group is a divalent, unbranched, saturated hydrocarbon chain, having from 8 to 15 carbon atoms.

28. The method of claim 26, wherein the spacer group is a 12 carbon methylene chain.

29. The method of any one of claims 1-7, wherein the capture reagent comprises a compound of formula (I):
A-B-C (I)
wherein:
A is a capture reagent oligonucleotide;
B is a spacer group; and
C is a linking group.

30. The method of claim 29, wherein the linking group is an amide, amine, carboxylic acid, alcohol or mercapto acid group.

31. The method of claim 29, wherein the spacer group is a divalent, unbranched, saturated hydrocarbon chain, having from 8 to 15 carbon atoms.

32. The method of any one of claims 1-7, wherein the competition oligonucleotide is about 15 to about 40 nucleotides in length.

33. The method of any one of claims 1-7, wherein the substrate is a chromogenic substrate.

34. The method of any one of claims 1-7, wherein the substrate is a dye or fluorophore.

35. The method of any one of claims 1-7, wherein the enzyme is horseradish peroxidase.

36. The method of claim 35, wherein the substrate is selected from:

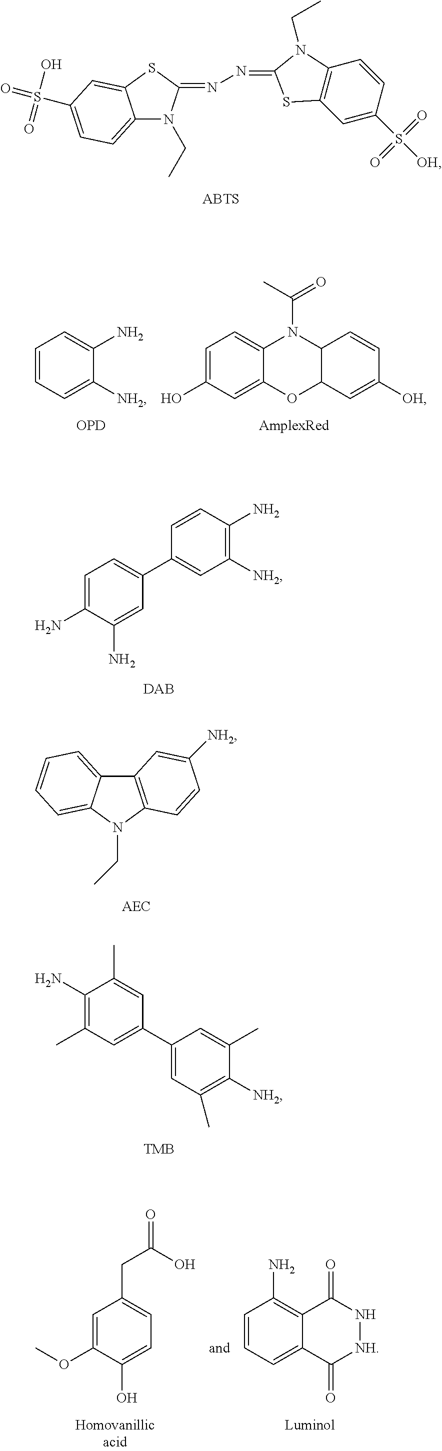

37. The method of any one of claims 1-7, wherein the enzyme is alkaline phosphatase (AP).

38. The method of claim 37, wherein the substrate is PNPP (p-nitrophenyl phosphate, disodium salt).

39. The method of any one of claims 1-7, wherein the enzyme is beta-galactosidase (β-gal).

40. The method of claim 39, wherein the substrate is ONPG (o-nitrophenyl-β-D-galactosidase), Nap-Gal (Naphthol-AS-Bl-β-D-galactosidase) or Mum-gal (4 methyl-umbelliferyl-β-D-galactosidase).

41. The method of any one of claims 1-7, wherein the concentration of the test oligonucleotide in the test sample is less than about 1 nanomole.

42. The method of any one of claims 1-7, wherein the concentration of the test oligonucleotide in the test sample is less than about 0.1 picomole.

43. The method of claim 17, wherein the vivo-morpholino comprises a phosphorodiamidate morpholino backbone and octaguanidine derivatizations.

44. The method of claim 43, wherein the vivo-morpholino comprises a sequence selected from:
(SEQ ID NO: 4)
5′-AAAAAGCCCTTCTATTGAAACACAGATACAAAA-3′
and
(SEQ ID NO: 7)
5′-AAAAAGCCCTTCTATTAAAACACAGACACAAAA-3′.

45. The method of any one of claims 1-7, wherein the capture reagent comprises a compound of formula (I):
A-B-C (I)
wherein:
A is a capture reagent oligonucleotide comprising a sequence having at least about 90% sequence identity to:
(SEQ ID NO: 5)
5′-TTTTGTATCTGTGTTTCAATAGAAGGGCTTTTT-3′
or
(SEQ ID NO: 8)
5′-TTTTGTGTCTGTGTTTTAATAGAAGGGCTTTTT-3′;
B is a 12 carbon methylene chain; and
C is an amine group.

46. The method of any one of claims 1-7, wherein the competition oligonucleotide comprises a sequence have at least about 90% sequence identity to:
5′-AAAAAGCCCTTCTATTGAAACACAGATACAAAA-3′ (SEQ ID NO:6) or
5′-AAAAAGCCCTTCTATTAAAACACAGACACAAAA-3′ (SEQ ID NO:9), and wherein the competition oligonucleotide is operably linked to horseradish peroxidase.

47. A capture reagent comprising a compound of formula (I):
A-B-C (I)
wherein:
A is a capture reagent oligonucleotide comprising a sequence having at least about 90% sequence identity to:
(SEQ ID NO: 5)
5′-TTTTGTATCTGTGTTTCAATAGAAGGGCTTTTT-3′
or
(SEQ ID NO: 8)
5′-TTTTGTGTCTGTGTTTTAATAGAAGGGCTTTTT-3′;
B is spacer group; and
C is a linking group.

48. The capture reagent of claim 47, wherein the spacer group is a divalent, unbranched, saturated hydrocarbon chain, having from 8 to 15 carbon atoms.

49. The capture reagent of claim 47, wherein the spacer group is a 12 carbon methylene chain.

50. The capture reagent of any one of claims 47-49, wherein linking group is an amide, amine, carboxylic acid, alcohol or mercapto acid group.

51. The capture reagent of any one of claims 47-49, wherein linking group is an amine group.

52. A competition oligonucleotide comprising a sequence having at least about 90% sequence identity to AAAAAGCCCTTCTATTGAAACACAGATACAAAA (SEQ ID NO:6) or AAAAAGCCCTTCTATTAAAACACAGACACAAAA (SEQ ID NO:9), which is operably linked to an enzyme.

53. The competition oligonucleotide of claim 52, wherein the enzyme is horseradish peroxidase (HRP), alkaline phosphatase (AP) or beta-galactosidase (β-gal).

54. A kit for detecting and/or quantifying a test oligonucleotide in a test sample comprising:
a) a capture reagent, wherein the capture reagent comprises an oligonucleotide comprising a nucleic acid sequence complementary to the test oligonucleotide;
b) a competition oligonucleotide operably linked to an enzyme, wherein the competition oligonucleotide comprises a nucleic acid sequence complementary to the capture oligonucleotide;
c) a standard test oligonucleotide(s) for calibration;
d) a substrate, wherein the substrate is capable of specifically binding to the enzyme to generate an enzyme-substrate reaction product, and wherein the enzyme-substrate reaction product is detectable spectrophotometrically or fluorometrically; and
e) instructions for use.

55. The kit of claim 54, comprising the capture reagent of any of claims 47-51.

56. The kit of claim 54 or 55, comprising the competition oligonucleotide of claim 52 or 53.
